(19) United States
(12) Patent Application Publication
(10) Pub. No.: US 2011/0034557 A1
(43) Pub. Date: Feb. 10, 2011

(54) ANTIMICROBIAL COMPOSITIONS AND USES THEREOF

(75) Inventors: Kevin A. Jarrell, Lincoln, MA (US); Gabriel Reznik, Waltham, MA (US)

Correspondence Address: CHOATE, HALL & STEWART LLP;Patent Department, Two International Place, Boston, MA, 02110, US

(73) Assignee: MODULAR GENETICS, INC., CAMBRIDGE, MA (US)

(21) Appl. No.: 12/709,270

(22) Filed: Feb. 19, 2010

Related U.S. Application Data
(60) Provisional application No. 61/154,313, filed on Feb. 20, 2009.

(51) Int. Cl. A61K 31/198; A61P 31/04; A01N 37/46; A01P 1/00

(52) U.S. Cl. 514/563

(57) ABSTRACT

Antimicrobial compositions and methods of using the compositions are described herein. The compositions include an antibacterial acyl amino acid. In some embodiments, the acyl amino acid is a fatty acylated glutamate. The methods herein include methods of using acyl amino acids for treating and preventing bacterial infections.

### CROSS-REFERENCE TO RELATED APPLICATIONS

The present application is copending with, shares at least one common inventor with and claims priority to U.S. provisional patent application Ser. No. 61/154,313, filed Feb. 20, 2009. The entire contents of the prior application are herein incorporated by reference.

### BACKGROUND

Bacterial pathogens such as Staphylococcus aureus are a major cause of human and animal disease worldwide. Evolving antibiotic resistance of bacterial pathogens provides a continual challenge in the prevention and clinical management of infections. S. aureus is a gram-positive bacteria often found on the skin or nose of humans as a commensal organism. Breach of protective barriers, e.g., due to surgery or injury, can lead to pathogenic effects. S. aureus infection is a major cause of morbidity and mortality in hemodialysis patients. S. aureus is also associated with post-surgical wound infection and sepsis.

### SUMMARY

Acyl amino acids having antimicrobial activity, compositions including the acyl amino acids, and uses of the acyl amino acids are provided herein.

Accordingly, in one aspect, the present disclosure provides a method of treating a microbial (e.g., bacterial) infection in a subject by administering a therapeutically effective amount of a pharmaceutical composition comprising an antimicrobial (e.g., antibacterial) acyl amino acid, wherein the acyl amino acid comprises a fatty acid.

In some embodiments, a bacterial infection comprises an infection by a Gram-positive bacteria. In some embodiments, a bacterial infection comprises a Staphylococcus infection, e.g., an infection by S. aureus. In some embodiments, an acyl amino acid comprises acyl glutamate. In some embodiments, the acyl amino acid comprises beta hydroxyl myristoyl glutamate.

In another aspect, the present disclosure provides a method of reducing risk for bacterial infection in a subject. The method includes administering to the subject a pharmaceutical composition comprising an antibacterial acyl amino acid, wherein the acyl amino acid comprises a fatty acid, and wherein the acyl amino acid is present in an amount sufficient to kill bacteria in the subject. In some embodiments, a subject is a subject at risk for exposure to Staphylococcus aureus. In some embodiments, a subject is at risk for nosocomial infection by Staphylococcus aureus. In some embodiments, an acyl amino acid comprises acyl glutamate. In some embodiments, an acyl amino acid comprises beta hydroxyl myristoyl glutamate.

In a further aspect, the present disclosure provides a composition (e.g., a pharmaceutical composition) comprising an antibacterial amino acid (e.g., acyl glutamate) and a carrier (e.g., a sterile carrier, e.g., a pharmaceutically acceptable carrier). In some embodiments, the antibacterial amino acid is acyl glutamate, wherein the acyl glutamate comprises a beta-hydroxy fatty acid, and wherein the acyl glutamate is present in an amount sufficient to kill S. aureus.

In a further aspect, the present disclosure provides a disinfectant composition comprising an antibacterial amino acid (e.g., acyl glutamate) and a carrier, wherein the acyl amino acid is present in an amount sufficient to kill a bacteria (e.g., S. aureus). In some embodiments, an acyl glutamate comprises a beta hydroxy fatty acid. In some embodiments, an acyl amino acid comprises beta hydroxyl myristoyl glutamate.

In another aspect, the present disclosure provides methods of producing an antibacterial composition. In some embodiments, methods include providing an acyl amino acid produced in a bacterial host cell which is engineered to produce the acyl amino acid, and forming a composition including the acyl amino acid (e.g., by combining the acyl amino acid with a carrier). In some embodiments, an engineered bacterial host cell is a Bacillus cell, e.g., a B. subtilis cell. In some embodiments, an acyl amino acid is acyl glutamate.

The details of one or more embodiments of the present disclosure are set forth in the description below. Other features, objects, and advantages of the present disclosure will be apparent from the description and from the claims. All cited patents, and patent applications and references (including references to public sequence database entries) are incorporated by reference in their entireties for all purposes.

### DESCRIPTION OF CERTAIN EMBODIMENTS

### Definitions

Acyl amino acid: The term “acyl amino acid” as used herein refers to an amino acid that is covalently linked to a fatty acid. In certain embodiments, acyl amino acids produced by compositions and methods of the present disclosure comprise a beta-hydroxy fatty acid. In certain embodiments, acyl amino acids are produced in engineered cells (e.g., Bacillus cells) that express engineered polypeptides comprising a peptide synthetase domain covalently linked to a fatty acid linkage domain (e.g., a beta-hydroxy fatty acid linkage domain) and a thioesterase domain or reductase domain. Typically, the identity of the amino acid moiety of the acyl amino acid is determined by the amino acid specificity of the peptide synthetase domain.

In some embodiments, an acyl amino acid includes a naturally occurring amino acid. In some embodiments, an acyl amino acid includes a non-naturally occurring amino acid, e.g. a modified amino acid. Examples of non-natural amino acids include, ornithine, citrulline, hydroxyproline, homoserine, phenylglycine, taurine, iodotyrosine, 2,4-diaminobutyric acid, alpha-amino isobutyric acid, 4-aminobutyric acid, 2-amino butyric acid, gamma-amino butyric acid, epsilon-amino hexanoic acid, 6-amino hexanoic acid, 2-amino isobutyric acid, 3-amino propionic acid, norleucine, norvaline, sarcosine, homocitrulline, cysteic acid, tau-butylglycine, tau-butylalanine, phenylglycine, cyclohexylalanine, beta-alanine, fluoro-amino acids, beta-methyl amino acids, C-methyl amino acids, N-methyl amino acids, and amino acid analogs. Non-natural amino acids also include amino acids having derivatized side groups.

Amino acids can be of D-(dextrorotary) form and/or L-(levorotary) forms. Acyl amino acids provided herein include free, salt, and ester forms. Salts can be, e.g., acid addition salts or base addition salts. In most embodiments, a salt is pharmaceutically acceptable. In some embodiments, a salt is prepared from an inorganic acid, e.g., hydrochloric, hydrobromic, hydroiodic, nitric, carbonic, sulfuric or phosphoric acid. in some embodiments, a salt is prepared from an organic acid, e.g., an aliphatic, cycloaliphatic, aromatic, arylaliphatic, heterocyclic, carboxylic or sulfonic organic acid, e.g., formic, acetic, propionic, succinic, glycolic, gluconic, maleic, embonic (pamoic), methanesulfonic, ethanesulfonic, 2-hydroxyethanesulfonic, pantothenic, benzenesulfonic, toluenesulfonic, sulfanilic, mesylic, cyclohexylaminosulfonic, stearic, algenic, β-hydroxybutyric, malonic, galactic, and galacturonic acid. In some embodiments, a salt is a metallic salt, e.g., an aluminum, calcium, lithium, magnesium, potassium, sodium or zinc salt. In some embodiments, a salt is an organic salt made from N₃N′-dibenzylethylenediamine, chloroprocaine, choline, diethanolamine, ethylenediamine, N-methylglucamine, lysine or procaine.

A beta-hydroxy fatty acid may be any of a variety of naturally occurring or non-naturally occurring beta-hydroxy fatty acids. In certain embodiments, an acyl amino acid of the present disclosure comprises a surfactant such as, without limitation, myristoyl glutamate.

Antibacterial: The term “antibacterial” as used herein to refer to an activity that inhibits bacterial cells. In some embodiments, an antibacterial compound causes reduction in viability and/or growth of bacterial cells (e.g., the compound is toxic to bacterial cells). In some embodiments, an antibacterial acyl amino acid is an amino acid that, when contacted with bacterial cells, causes a reduction in the number and/or growth of the cells. In some embodiments, an antibacterial acyl amino acid is antibacterial to S. aureus cells.

Antimicrobial: The term “antimicrobial” as used herein to refer to an activity that inhibits microbial cells. In some embodiments, an antimicrobial compound causes reduction in viability and/or growth of microbial cells (e.g., the compound is toxic to microbial cells). In some embodiments, an antimicrobial acyl amino acid is an amino acid that, when contacted with microbial cells, causes a reduction in the number and/or growth of the cells. In some embodiments, an antimicrobial acyl amino acid is antibacterial to S. aureus cells.

Beta-hydroxy fatty acid linkage domain: The term “beta-hydroxy fatty acid linkage domain” as used herein refers to a polypeptide domain that covalently links a beta-hydroxy fatty acid to an amino acid to form an acyl amino acid. In certain embodiments, a beta-hydroxy fatty acid linkage domain is covalently linked to a peptide synthetase domain and a thioesterase domain to generate an engineered polypeptide useful in the synthesis of an acyl amino acid. In certain embodiments, a beta-hydroxy fatty acid linkage domain is covalently linked to a peptide synthetase domain and a reductase domain to generate an engineered polypeptide useful in the synthesis of an acyl amino acid. A variety of beta-hydroxy fatty acid linkage domains are known to those skilled in the art. However, different beta-hydroxy fatty acid linkage domains often exhibit specificity for one or more beta-hydroxy fatty acids. As one non-limiting example, a beta-hydroxy fatty acid linkage domain from surfactin synthetase is specific for the beta-hydroxy myristic acid, which contains 13 to 15 carbons in the fatty acid chain. Thus, the beta-hydroxy fatty acid linkage domain from surfactin synthetase can be used to construct an engineered polypeptide useful in the generation of an acyl amino acid that comprises a fatty acid beta-hydroxy myristic acid.

Beta-hydroxy fatty acid: The term “beta-hydroxy fatty acid” as used herein refers to a fatty acid chain comprising a hydroxy group at the beta position of the fatty acid chain. As is understood by those skilled in the art, the beta position corresponds to the third carbon of the fatty acid chain, the first carbon being the carbon of the carboxylate group. Thus, when used in reference to an acyl amino acid, where the carboxylate moiety of the fatty acid has been covalently attached to the nitrogen of the amino acid, the beta position corresponds to the carbon two carbons removed from the carbon having the ester group. A beta-hydroxy fatty acid may contain any number of carbon atoms in the fatty acid chain. As non-limiting examples, a beta-hydroxy fatty acid may contain 3, 4, 5, 6, 7, 8,9, 10, 11, 12, 3, 14, 15, 15, 16, 17, 18, 19, 20 or more carbon atoms. Beta-hydroxy fatty acids may contain linear carbon chains, in which each carbon of the chain, with the exception of the terminal carbon atom and the carbon attached to the nitrogen of the amino acid, is directly covalently linked to two other carbon atoms. Additionally or alternatively, beta-hydroxy fatty acids may contain branched carbon chains, in which at least one carbon of the chain is directly covalently linked to three or more other carbon atoms. Beta-hydroxy fatty acids may contain one or more double bonds between adjacent carbon atoms. Alternatively, beta-hydroxy fatty acids may contain only single-bonds between adjacent carbon atoms. A non-limiting exemplary beta-hydroxy fatty acid is beta-hydroxy myristic acid, which contains 13 to 15 carbons in the fatty acid chain. Different beta-hydroxy fatty acid linkage domains that exhibit specificity for other beta-hydroxy fatty acids (e.g., naturally or non-naturally occurring beta-hydroxy fatty acids) may be used to generate an acyl amino acid of the practitioner's choosing.

Domain, Polypeptide domain: The terms “domain” and “polypeptide domain” as used herein generally refer to polypeptide moieties that naturally occur in longer polypeptides, or to engineered polypeptide moieties that are homologous to such naturally occurring polypeptide moieties, which polypeptide moieties have a characteristic structure (e.g., primary structure such as the amino acid sequence of the domain, although characteristic structure of a given domain also encompasses secondary, tertiary, quaternary, etc. structures) and exhibit one or more distinct functions. As will be understood by those skilled in the art, in many cases polypeptides are modular and are comprised of one or more polypeptide domains, each domain exhibiting one or more distinct functions that contribute to the overall function of the polypeptide. The structure and function of many such domains are known to those skilled in the art. For example, Fields and Song (Nature, 340(6230): 245-6, 1989) showed that transcription factors are comprised of at least two polypeptide domains: a DNA binding domain and a transcriptional activation domain, each of which contributes to the overall function of the transcription factor to initiate or enhance transcription of a particular gene that is under control of a particular promoter sequence. A polypeptide domain, as the term is used herein, also refers an engineered polypeptide that is homologous to a naturally occurring polypeptide domain. “Homologous”, as the term is used herein, refers to the characteristic of being similar at the nucleotide or amino acid level to a reference nucleotide or polypeptide. For example, a polypeptide domain that has been altered at one or more positions such that the amino acids of the reference polypeptide have been substituted with amino acids exhibiting similar biochemical characteristics (e.g., hydrophobicity, charge, bulkiness) will generally be homologous to the reference polypeptide. Percent identity and similarity at the nucleotide or amino acid level are often useful measures of whether a given nucleotide or polypeptide is homologous to a reference nucleotide or amino acid. Those skilled in the art will understand the concept of homology and will be able to determine whether a given nucleotide or amino acid sequence is homologous to a reference nucleotide or amino acid sequence.

Dosing Regimen: A “dosing regimen”, as that term is used herein, refers to a set of unit doses (typically more than one) that are administered individually separated by periods of time. The recommended set of doses (i.e., amounts, timing, route of administration, etc.) for a particular pharmaceutical agent constitutes its dosing regimen.

Engineered: The term “engineered” as used herein refers to a non-naturally occurring moiety that has been created by the hand of man. For example, in reference to a polypeptide, an “engineered polypeptide” refers to a polypeptide that has been designed and/or manipulated to comprise a polypeptide that does not exist in nature. In various embodiments, an engineered polypeptide comprises two or more covalently linked polypeptide domains. Typically such domains will be linked via peptide bonds, although the present disclosure is not limited to engineered polypeptides comprising polypeptide domains linked via peptide bonds, and encompasses other covalent linkages known to those skilled in the art. One or more covalently linked polypeptide domains of engineered polypeptides may be naturally occurring. Thus, in certain embodiments, engineered polypeptides comprise two or more covalently linked domains, at least one of which is naturally occurring. In certain embodiments, two or more naturally occurring polypeptide domains are covalently linked to generate an engineered polypeptide. For example, naturally occurring polypeptide domains from two or more different polypeptides may be covalently linked to generate an engineered polypeptide. In certain embodiments, naturally occurring polypeptide domains of an engineered polypeptide are covalently linked in nature, but are covalently linked in the engineered polypeptide in a way that is different from the way the domains are linked nature. For example, two polypeptide domains that naturally occur in the same polypeptide but which are separated by one or more intervening amino acid residues may be directly covalently linked (e.g., by removing the intervening amino acid residues) to generate an engineered polypeptide. Additionally or alternatively, two polypeptide domains that naturally occur in the same polypeptide which are directly covalently linked together (e.g., not separated by one or more intervening amino acid residues) may be indirectly covalently linked (e.g., by inserting one or more intervening amino acid residues) to generate an engineered polypeptide. In certain embodiments, one or more covalently linked polypeptide domains of an engineered polypeptide may not exist naturally. For example, such polypeptide domains may be engineered themselves.

Fatty acid linkage domain: The term “fatty acid linkage domain” as used herein refers to a polypeptide domain that covalently links a fatty acid to an amino acid to form an acyl amino acid. In certain embodiments, a fatty acid linkage domain is covalently linked to a peptide synthetase domain and a thioesterase domain to generate an engineered polypeptide useful in the synthesis of an acyl amino acid. In certain embodiments, a fatty acid linkage domain is covalently linked to a peptide synthetase domain and a reductase domain to generate an engineered polypeptide useful in the synthesis of an acyl amino acid. A variety of fatty acids are known to those of ordinary skill in the art, as are a variety of fatty acid linkage domains, such as for example, fatty acid linkage domains present in various peptide synthetase complexes that produce lipopeptides. In certain embodiments, a fatty acid linkage domain of the present disclosure comprises a beta-hydroxy fatty acid linkage domain.

Naturally occurring: The term “naturally occurring”, as used herein when referring to an amino acid, refers to one of the standard group of twenty amino acids that are the building blocks of polypeptides of most organisms, including alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, and valine. In certain embodiments, the term “naturally occurring” also refers to amino acids that are used less frequently and are typically not included in this standard group of twenty but are nevertheless still used by one or more organisms and incorporated into certain polypeptides. For example, the codons UAG and UGA normally encode stop codons in most organisms. However, in some organisms the codons UAG and UGA encode the amino acids selenocysteine and pyrrolysine. Thus, in certain embodiments, selenocysteine and pyrrolysine are naturally occurring amino acids.

In combination: The phrase “in combination”, as used herein, refers to two or more agents that are simultaneously administered to a subject. It will be appreciated that two or more agents are considered to be administered “in combination” whenever a subject is simultaneously exposed to both (or more) of the agents. Each of the two or more agents may be administered according to a different schedule; it is not required that individual doses of different agents be administered at the same time, or in the same composition. Rather, so long as both (or more) agents are present (e.g., at relevant levels) in the subject's body, they are considered to be administered “in combination”.

Peptide synthetase complex: The term “peptide synthetase complex” as used herein refers to an enzyme that catalyzes the non-ribosomal production of a variety of peptides. A peptide synthetase complex may comprise a single enzymatic subunit (e.g., a single polypeptide), or may comprise two or more enzymatic subunits (e.g., two or more polypeptides). A peptide synthetase complex typically comprises at least one peptide synthetase domain, and may further comprise one or more additional domains such as for example, a fatty acid linkage domain, a thioesterase domain, a reductase domain, etc. Peptide synthetase domains of a peptide synthetase complex may comprise two or more enzymatic subunits, with two or more peptide synthetase domains present in a given enzymatic subunit. For example the surfactin peptide synthetase complex (also referred to herein simply as “surfactin synthetase complex”) comprises three distinct polypeptide enzymatic subunits: the first two subunits comprise three peptide synthetase domains, while the third subunit comprises a single peptide synthetase domain.

Peptide synthetase domain: The term “peptide synthetase domain” as used herein refers to a polypeptide domain that minimally comprises three domains: an adenylation (A) domain, responsible for selectively recognizing and activating a specific amino acid, a thiolation (T) domain, which tethers the activated amino acid to a cofactor via thioester linkage, and condensation (C) domain, which links amino acids joined to successive units of the peptide synthetase by the formation of amide bonds. A peptide synthetase domain typically recognizes and activates a single, specific amino acid, and in the situation where the peptide synthetase domain is not the first domain in the pathway, links the specific amino acid to the growing peptide chain. In certain embodiments, a peptide synthetase domain is covalently linked to a fatty acid linkage domain such as a beta-hydroxy fatty acid linkage domain and a thioesterase domain, which construct may be advantageously used to generate an acyl amino acid. In certain embodiments, a peptide synthetase domain is covalently linked to a fatty acid linkage domain such as a beta-hydroxy fatty acid linkage domain and a reductase domain, which construct may be advantageously used to generate an acyl amino acid. A variety of peptide synthetase domains are known to those skilled in the art, e.g. such as those present in a variety of nonribosomal peptide synthetase complexes. Those skilled in the art will be aware of methods to determine whether a give polypeptide domain is a peptide synthetase domain. Different peptide synthetase domains often exhibit specificity for one or more amino acids. As one non-limiting example, the first peptide synthetase domain from the surfactin synthetase Srf-A subunit is specific for glutamate. Thus, the peptide synthetase domain from surfactin synthetase can be used in accordance with the present disclosure to construct an engineered polypeptide useful in the generation of an acyl amino acid that comprises the amino acid glutamate. Different peptide synthetase domains that exhibit specificity for other amino acids (e.g., naturally or non-naturally occurring amino acids) may be used in accordance with the present disclosure to generate any acyl amino acid of the practitioner's choosing.

Polypeptide: The term “polypeptide” as used herein refers to a series of amino acids joined together in peptide linkages, such as polypeptides synthesized by ribosomal machinery in naturally occurring organisms. The term “polypeptide” also refers to a series of amino acids joined together by non-ribosomal machinery, such as by way of non-limiting example, polypeptides synthesized by various peptide synthetases. Such non-ribosomally produced polypeptides exhibit a greater diversity in covalent linkages than polypeptides synthesized by ribosomes (although those skilled in the art will understand that the amino acids of ribosomally-produced polypeptides may also be linked by covalent bonds that are not peptide bonds, such as the linkage of cystines via di-sulfide bonds). For example, surfactin is a lipopeptide synthesized by the surfactin synthetase complex. Surfactin comprises seven amino acids, which are initially joined by peptide bonds, as well as a beta-hydroxy fatty acid covalently linked to the first amino acid, glutamate. However, upon addition the final amino acid (leucine), the polypeptide is released and the thioesterase domain of the SRFC protein catalyzes the release of the product via a nucleophilic attack of the beta-hydroxy of the fatty acid on the carbonyl of the C-terminal Leu of the peptide, cyclizing the molecule via formation of an ester, resulting in the C-terminus carboxyl group of leucine attached via a lactone bond to the b-hydroxyl group of the fatty acid. Polypeptides can be two or more amino acids in length, although most polypeptides produced by ribosomes and peptide synthetases are longer than two amino acids. For example, polypeptides may be 2, 3, 4, 5, 6, 7, 8,9, 10, 15, 20, 25, 30, 35, 40, 45, 50, 55, 60, 65, 70, 75, 80, 85, 90, 95, 100, 150, 200, 250, 300, 350, 400, 450, 500 or more amino acids in length.

Reductase Domain: The term “reductase domain” as used herein refers to a polypeptide domain that catalyzes release of an acyl amino acid produced by a peptide synthetase complex from the peptide synthetase complex. In certain embodiments, a reductase domain is covalently linked to a peptide synthetase domain and a fatty acid linkage domain such as a beta-hydroxy fatty acid linkage domain to generate an engineered polypeptide useful in the synthesis of an acyl amino acid. A variety of reductase domains are found in nonribosomal peptide synthetase complexes from a variety of species. A non-limiting example of a reductase domain that may be used in accordance with the present disclosure includes the reductase domain from linear gramicidin (ATCC8185). However, any reductase domain that releases an acyl amino acid produced by a peptide synthetase complex from the peptide synthetase complex may be used in accordance with the present disclosure. Reductase domains are characterized by the presence of the consensus sequence: [LIVSPADNK]-x(9)-{P}-x(2)-Y-[PSTAGNCV]-[STAGNQCIVM]-[STAGC]-K-{PC}-[SAGFYR]-[LIVMSTAGD]-x-{K}-[LIVMFYW]-{D}-x-{YR}-[LIVMFYWGAPTHQ]-[GSACQRHM] (SEQ ID NO: 1), where square brackets (“[ ]”) indicate amino acids that are typically present at that position, squiggly brackets (“{ }”) indicate amino acids that amino acids that are typically not present at that position, and “x” denotes any amino acid or a gap. X(9) for example denotes any amino acids or gaps for nine consecutive positions. Those skilled in the art will be aware of methods to determine whether a give polypeptide domain is a reductase domain.

Subject: As used herein, the term “subject” or “patient” refers to any cell or organism to which a composition of the present disclosure may be administered, e.g., for experimental, diagnostic, prophylactic, and/or therapeutic purposes, and/or from which a sample may be obtained. In some embodiments, subjects include animals (e.g., mammals such as mice, rats, rabbits, non-human primates, cows, pigs, and humans; birds, e.g., chickens; insects; worms; etc.). In some embodiments, a subject is a human. In some embodiments, a subject is a plant. In some embodiments, a subject is a cell culture. In some embodiments, a subject has a bacterial infection. In some embodiments, a subject is at risk for a bacterial infection (e.g., the subject is immunodeficient and/or is at risk for a nosocomial infection).

Therapeutically effective amount: “Therapeutically effective amount” refers to an amount of an agent (e.g., an acyl amino acid) that inhibits and/or delays the onset, alleviates the symptoms, or controls an infection, e.g., a bacterial infection. A therapeutic effect may be objective (i.e., measurable by some test or marker) or subjective (i.e., subject gives an indication of or feels an effect). A therapeutically effective amount is commonly administered in a dosing regimen that may comprise multiple unit doses. For any particular pharmaceutical agent, a therapeutically effective amount (and/or an appropriate unit dose within an effective dosing regimen) may vary, for example, depending on route of administration, on combination with other pharmaceutical agents. Also, the specific therapeutically effective amount (and/or unit dose) for any particular subject may depend upon a variety of factors including the condition (e.g., infection) being treated and the severity of the condition; the activity of the specific pharmaceutical agent employed; the specific composition employed; the age, body weight, general health, sex and diet of the subject; the time of administration, route of administration, and/or rate of excretion or metabolism of the specific pharmaceutical agent employed; the duration of the treatment; and like factors as is well known in the medical arts.

Thioesterase domain: The term “thioesterase domain” as used herein refers to a polypeptide domain that catalyzes release of an acyl amino acid produced by a peptide synthetase complex from the peptide synthetase complex. In certain embodiments, a thioesterase domain is covalently linked to a peptide synthetase domain and a fatty acid linkage domain such as a beta-hydroxy fatty acid linkage domain to generate an engineered polypeptide useful in the synthesis of an acyl amino acid. A variety of thioesterase domains are found in nonribosomal peptide synthetase complexes from a variety of species. A non-limiting example of a thioesterase domain that may be used in accordance with the present disclosure includes the thioesterase domain from the Bacillus subtilis surfactin synthetase complex, present in Srf-C subunit. However, any thioesterase domain that releases an acyl amino acid produced by a peptide synthetase complex from the peptide synthetase complex may be used in accordance with the present disclosure. Thioesterase domains are characterized by the presence of the consensus sequence: [LIV]-{KG}-[LIVFY]-[LIVMST]-G-[HYWV]-S-{YAG}-G-[GSTAC] (SEQ ID NO: 2), where square brackets (“[ ]”) indicate amino acids that are typically present at that position, and squiggly brackets (“{ }”) indicate amino acids that amino acids that are typically not present at that position. Those skilled in the art will be aware of methods to determine whether a give polypeptide domain is a thioesterase domain.

Treating: “Treating” or “treatment” refers to both therapeutic treatment and prophylactic or preventative measures, wherein the object is to prevent or alleviate a microbial infection or a symptom thereof. In some embodiments, a subject is successfully “treated” for an infection if, after receiving a therapeutically effective amount of a composition (e.g., a composition comprising an acyl amino acid), the subject shows an observable and/or measurable reduction one or more of microbial burden, a symptom of microbial infection, and/or relief to some extent, of one or more of the symptoms associated with a microbial infection, and/or reduced morbidity and mortality.

Unit dose: The term “unit dose”, as used herein, refers to a discrete administration of a pharmaceutical agent, typically in the context of a dosing regimen.

### Acyl Amino Acids

The present disclosure encompasses the recognition that acyl amino acids have antimicrobial activity and can be used, inter alia, in pharmaceutical, disinfectant, and other types of compositions to inhibit microbial growth and/or treat infections. In some embodiments, acyl amino acids have antimicrobial activity against Gram-positive bacteria such as S. aureus. Any of a variety of acyl amino acids may be employed in the compositions and methods of the present disclosure. In certain embodiments, acyl amino acids provided herein comprise an amino acid selected from one of the twenty amino acids commonly employed in ribosomal peptide synthesis. Thus, acyl amino acids of may comprise alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, and/or valine. In certain embodiments, acyl amino acids comprise amino acids other than these twenty. For example, acyl amino acids may comprise amino acids used less commonly during ribosomal polypeptide synthesis such as, without limitation, selenocysteine and/or pyrrolysine. In certain embodiments, acyl amino acids comprise amino acids that are not used during ribosomal polypeptide synthesis such as, without limitation, norleucine, beta-alanine and/or ornithine, and/or D-amino acids. In certain embodiments, acyl amino acids include glutamic acid.

Acyl amino acids of compositions and methods of the present disclosure comprise a fatty acid moiety. A fatty acid of acyl amino acids of the present disclosure may be any of a variety of fatty acids known to those of ordinary skill in the art. Fatty acids can be varied in length, branching, and/or degree of saturation. For example, a fatty acid can contain 3, 4, 5, 6, 7, 8, 9, 10, 11, 12, 13, 14, 15, 16, 17, 18, 19, 20, or more carbon atoms. A fatty acid may contain linear carbon chains and/or branched chains. A fatty acid may contain one or more double bonds between adjacent carbon atoms. Alternatively, a fatty acid may contain only single bonds between adjacent carbon atoms. In some embodiments, a fatty acid chain includes 13-15 carbon atoms.

Acyl amino acids present in compositions provided herein can include amino acids and/or fatty acids of homogeneous or heterogeneous structure. In some embodiments, compositions include a single type of amino acid (e.g., glutamate), and a single type of fatty acid chain. In some embodiments, compositions include a single type of amino acid (e.g., glutamate), and fatty acid chains of varying lengths. In some embodiments, compositions include multiple types of amino acids and single or multiple types of fatty acid chains. In some embodiments, acyl amino acid compositions are produced in engineered microbial host cells (e.g., bacterial cells, such as Bacillus cells, e.g., B. subtilis cells), and include fatty acid chains of heterogeneous lengths. Such heterogeneity can be characteristic of the host cells.

Acyl amino acids of the present disclosure may comprise saturated fatty acids such as, without limitation, butryic acid, caproic acid, caprylic acid, capric acid, lauric acid, myristic acid, palmitic acid, stearic arachidic acid, behenic acid, and/or lignoceric acid. In certain embodiments, acyl amino acids of the present disclosure may comprise unsaturated fatty acids such as, without limitation, myristoleic acid, palmitoleic acid, oliec acid, linoleic acid, alpha-linolenic acid, arachidonic acid, eicosapentaenoic acid, erucic acid, and/or docosahexaenoic acid. Other saturated and unsaturated fatty acids that may be part of an acyl amino acid in accordance with the present disclosure will be known to those of ordinary skill in the art. In certain embodiments, acyl amino acids of the present disclosure comprise beta-hydroxy fatty acids as the fatty acid moiety. As is understood by those of ordinary skill in the art, beta-hydroxy fatty acids comprise a hydroxy group attached to the third carbon of the fatty acid chain, the first carbon being the carbon of the carboxylate group. In certain embodiments, an acyl amino acid comprises beta hydroxy myristic acid. In certain embodiments, an acyl amino acid is beta hydroxyl myristoyl glutamate.

Methods for characterizing antimicrobial effects of compounds in vitro and in vivo are known and can be used to evaluate antimicrobial activity of acyl amino acids described herein. In some embodiments, antibacterial activity of an acyl amino acid is evaluated in vitro. In some embodiments, antibacterial activity of an acyl amino acid is evaluated in vivo, e.g., using a mouse model of bacterial infection. In one example for evaluating activity toward S. aureus, mice are injected with a lethal dose of S. aureus and administered a composition containing an acyl amino acid at varying doses (e.g., 1-10 mg/kg) just after infection and again at 5 hours after infection. Survival of animals treated with the composition is compared to controls.

### Generation of Acyl Amino Acids

Acyl amino acids can be produced by synthetic means and/or by expression in microorganisms. In certain embodiments, acyl amino acids are produced in engineered microorganisms (e.g., engineered to produce an acyl amino acid of interest). In certain embodiments, acyl amino acid compositions are produced with engineered polypeptides. Engineered polypeptides for producing acyl amino acids can include, for example, a peptide synthetase domain covalently linked to a fatty acid linkage domain and a thioesterase domain. In certain embodiments, engineered polypeptides for producing acyl amino acids include a peptide synthetase domain covalently linked to a beta-hydroxy fatty acid linkage domain and a thioesterase domain. In certain embodiments, engineered polypeptides include a peptide synthetase domain covalently linked to a fatty acid linkage domain and a reductase domain. In certain embodiments, engineered polypeptides include a peptide synthetase domain covalently linked to a beta-hydroxy fatty acid linkage domain and a reductase domain. Engineered polypeptides useful for producing acyl amino acids are described in PCT/US08/60474, published as WO2008/131002, which is incorporated herein by reference in its entirety.

In certain embodiments, acyl amino acids are produced in Bacillus, e.g., B. subtilis.

Acyl amino acids produced in microorganisms can be recovered by any available means. In some embodiments, acyl amino acids are recovered from culture media by filtration and HPLC purification (e.g., on a C18 column). In some embodiments, acyl amino acids are subjected to extraction (e.g., with a solvent such as butanol), e.g., to remove impurities such as salts and/or proteins.

Acyl amino acids can be produced synthetically. In some embodiments, an acyl amino acid is produced by an acylation reaction of an amino acid and a fatty acid, fatty acid ester, or fatty acid chloride. See, e.g., Takehara et al., J. Am. Oil Chem. Soc. 49:134, 1972.

### Acyl Amino Acid Compostions and Uses Thereof

The present disclosure provides, inter alia, methods for treating microbial infections, e.g., bacterial infections, in a subject with a therapeutically-effective amount of an acyl amino acid composition. In some embodiments, a subject is a human or other animal in need of antimicrobial treatment. In some embodiments, a subject is a cell (e.g., a cell in cell culture).

A method can include administering to the subject an effective dose of a composition including an acyl amino acid. An effective dose can be between about 0.1 and about 100 mg/kg of an acyl amino acid. In some embodiments, a dose is from about 0.1 to about 50 mg/kg. In some embodiments, a dose is from about 1 to 25 mg/kg. In some embodiments, an effective dose for a cell in culture is between 0.1 and 1000 ug/mL, e.g., between 0.1 and 200 ug/mL.

An amount in an administered dose or the total amount administered will depend on various factors. In some embodiments, factors include nature and severity of an infection, age of a subject, health of a subject, tolerance of a subject to the compound and the microorganism or microorganisms involved in the infection.

A composition including an acyl amino acid can be administered as a single daily dose or in multiple doses per day. A course of treatment may require administration over extended periods of time, e.g., for several days or for from two to four weeks. In some embodiments, an acyl amino acid composition is administered for a period of time from 3 days to 6 months. In some embodiments, an acyl amino acid composition is administered for 7 to 56 days. In some embodiments, an acyl amino acid composition is administered for 7 to 28 days. In some embodiments, an acyl amino acid composition is administered for 7 to 14 days. In some embodiments, an acyl amino acid composition is administered until a microbial infection is eradicated or reduced.

An acyl amino acid composition can also be administered in food. In some embodiments in which a composition is administered as part of a total dietary intake, an amount of composition is less than 1% by weight of a diet, e.g., no more than 0.5% by weight. In some embodiments, an acyl amino acid is added to food. In some embodiments, an acyl amino acid is added to a premix.

Methods herein can include administering an acyl amino acid composition to a subject in need thereof in an amount that is efficacious in reducing (e.g., partially reducing or eliminating) a microbial infection. A composition may be administered orally, parenterally, by inhalation, topically, rectally, nasally, buccally, vaginally, or by an implanted reservoir, external pump or catheter. A composition can be prepared for ophthalmic or aerosolized uses. In some embodiments, a composition is administered as an aerosol. Any type of aerosol delivery system can be used. In some embodiments, an aerosol delivery vehicle is an anhydrous or dry powder inhaler. In some embodiments, an acyl amino acid composition is directly injected or administered into an abscess, ventricle or joint. Parenteral administration can include subcutaneous, intravenous, intramuscular, intra-articular, intra-synovial, cisternal, intrathecal, intrahepatic, intralesional and intracranial injection or infusion. In some embodiments, an acyl amino acid composition is administered intravenously, subcutaneously or orally

In some embodiments, an acyl amino acid composition is administered to a subject having an infection by a gram-positive bacteria. Gram-positive bacteria include, for example, Actinomyces spp., Bifidobacterium spp., Clostridium difficile, C. clostridiiforme, C. innocuum, C. perfringens, C. ramosum, Corynebacterium jeikeium, enterococci (e.g., vancomycin susceptible enterococci, and vancomycin-resistant strains such as Enterococcus faecalis and E. faecium), Escherichia spp. (e.g., E. coli), Eubacterium aerofaciens, E. lentum, Haemophilus influenzae, Lactobacillus acidophilus, L. casei, L. plantarum, Lactococcus spp., Leuconostoc spp., Listeria monocytogenes, Moraxella spp., Pediococcus, Peptostreptococcus anaerobius, P. asaccarolyticus, P. magnus, P. micros, P. prevotil, P. productus, Propionibacterium acnes, staphylococci, e.g., methicillin-susceptible and methicillin-resistant staphylococci (e.g., Staphylococcus aureus, S. epidermidis, S. haemolyticus, S. hominis, S. saprophyticus, and coagulase-negative staphylococci), glycopeptide intermediary-susceptible S. aureus (GISA), streptococci, e.g., penicillin-susceptible and penicillin-resistant streptococci (e.g., Streptococcus pneumoniae, S. pyogenes, S. agalactiae, S. avium, S. bovis, S. lactis, S. sangius and Streptococci Group C, and Streptococci Group G and viridans streptococci).

Methods employing acyl amino acid compositions can be used to treat an infection of any organ or tissue. In some embodiments, an acyl amino acid composition is used to treat an infection of one or more of skeletal muscle, skin, bloodstream, kidneys, heart, lung or bone. In some embodiments, an acyl amino acid composition is used to treat an infection of skin and/or soft tissue. In some embodiments, an acyl amino acid composition is used to treat bacteremia. In some embodiments, an acyl amino acid composition is used to treat a urinary tract infection. In some embodiments, an acyl amino acid composition is used to treat a wound (e.g., burned tissue, surgical wound, abrasion, ulceration, or other type of lesion). In some embodiments, an acyl amino acid composition is used to treat a respiratory infection, e.g., otitis media, sinusitis, chronic bronchitis and/or pneumonia, e.g., pneumonia caused by drug-resistant S. pneumoniae or H. influenzae. In some embodiments, an acyl amino acid composition is used to treat a mixed infection, e.g., an infection comprising different types of gram-positive bacteria, or comprising both gram-positive and gram-negative bacteria. In some embodiments, an acyl amino acid composition is used to treat an intra-abdominal infection or an obstetrical/gynecological infection. In some embodiments, an acyl amino acid composition is used to treat endocarditis, nephritis, septic arthritis, intra-abdominal sepsis, a bone infection, a joint infection, or osteomyelitis.

In some embodiments, an acyl amino acid composition is administered to a subject having a bacterial infection that is resistant to one or more other compounds, e.g., penicillin, methicillin, amoxocillin, oxacillin, or vancomycin. In some embodiments, an acyl amino acid composition is administered to a subject who has received, or is receiving antibiotic therapy for the infection for which the acyl amino acid composition is administered. In some embodiments, more than one type of acyl amino acid composition is used to treat a subject.

In some embodiments, an acyl amino acid composition is administered to a subject with a second antimicrobial agent. Antimicrobial agents that may be co-administered include, amikacin, aminoglycosides, Aztreonam, bacitracin, Biapenem, capreomycin, carbapenems, carumonam, Cefetamct pivoxil, Cefluprenam, Cefoselis, Cefozopran, Cefpirome, ceftriaxone, cephalosporins, chloramphenicol, clindamycin, cycloserine, Cyclothialidine, Dynemicin A, Epiroprim, ethambutol, ethionamide, eveminomicin, fluoroquinolones, fosfomycin, fusidate sodium, gentamicin, glycopeptide, glycylcycline, gramicidin, imipenen, isoniazid, ketolides, Kosan, Lenapenem, lincomycin, Linezolid, macrolides, Mersacidin, methenamine mandelate, methenamine hippurate, Metronidazole, mupirocin, micacocidin A, netilmicin, nitrofurans, nitroimidazoles, oxazolidinone, novobiocin, para-aminosalicylic acid (PAS), penicillins, polymyxins, prothionamide, pyrazinamide, pyrimethamine, quinolones, rifamycins, Rifalazil, ritipenam acoxyl, Sanfetrinem celexetil, Sanfetrinem sodium, spectinomycin, streptogramins, Sulopenem, Synercid, sulfonamides, teicoplanin, tetracyclines, thiacetazone, thiamphenicol, trimethoprim, vancomycin, Veneprim, viomycin, Ziracin, LY 333328, CL 331002, OCA-983, GV-143253, CS-834, A-99058.1, A-165600, A-179796, KA 159, DX8739, DU 6681; ER 35786, HGP-31, HMR-3647, RU-59863, KP 736,; AM 1732, MEN 10700, BO 2502A, NE-1530, PR 39, K130, OPC 20000, OPC 2045, PD 138312, PD 140248, CP 111905, RO-65-5788, Sch-40832, SEP-132613, SB-275833, SR-15402, SUN A0026, TOC 39, and T 3811.

The present disclosure provides antimicrobial (e.g., pharmaceutical) compositions and formulations including acyl amino acid compositions (which include compositions having a free acyl amino acid or salt thereof). In some embodiments, an antimicrobial composition includes an acyl amino acid as a sole active (antimicrobial) agent. In some embodiments, an antimicrobial composition includes an agent having antimicrobial activity, in addition to an antimicrobial acyl amino acid.

Pharmaceutical compositions comprising acyl amino acids be formulated for oral, intranasal, intravaginal, inhalation, intravenous, intramuscular, subcutaneous or parenteral administration for the therapeutic or prophylactic treatment of microbial infections, e.g., bacterial infections. In some embodiments for oral or parenteral administration, acyl amino acid compositions are mixed with one or more pharmaceutical carriers and/or excipients and used in the form of tablets, capsules, elixirs, suspensions, syrups, wafers, etc. Compositions can include from about 0.1 to about 99% by weight of the active compound (i.e., an acyl amino acid). In some embodiments, a composition includes about 10 to about 30% acyl amino acid. In certain embodiments, a composition includes about 20 to about 40% acyl amino acid. In certain embodiments, a composition includes about 5% acyl amino acid. In certain embodiments, a composition includes about 10% acyl amino acid. In certain embodiments, a composition includes about 20% acyl amino acid. In certain embodiments, a composition includes about 30% acyl amino acid. In certain embodiments, a composition includes about 40% acyl amino acid. In certain embodiments, a composition includes about 50% acyl amino acid. In certain embodiments, a composition includes about 60% acyl amino acid. In certain embodiments, a composition includes about 70% acyl amino acid. In certain embodiments, a composition includes about 80% acyl amino acid. In certain embodiments, a composition includes about 90% acyl amino acid. In certain embodiments, a composition includes about 95% acyl amino acid. Acyl amino acids provided herein can have surfactant properties. In certain embodiments, an acyl amino acid used for its antimicrobial properties is used at a concentration other than a concentration at which it is typically used as a surfactant, e.g., in a detergent solution. For example, in some embodiments, an acyl amino acid which is used at about 15-18% (total weight) in a detergent solution is present in an antimicrobial composition in an amount of at least 20%, 25%, 30%, 35%, 40%, 45%, 50%, 55%, 60%, 65%, 70%, or 75% (total weight).

A pharmaceutical composition can be prepared in accordance with standard procedures and are administered at dosages that are selected to reduce, prevent or eliminate an infection (See, e. g., Remington's Pharmaceutical Sciences, Mack Publishing Company, Easton, Pa. and Goodman and Gilman's. The Pharmaceutical Basis of Therapeutics, Pergamon Press, New York, N.Y., the contents of which are incorporated herein by reference, for a general description of the methods for administering various antimicrobial agents for human therapy). In some embodiments, compositions are delivered using controlled (e.g., capsules) or sustained release delivery systems (e.g., bioerodable matrices) (see, e.g., U.S. Pat. Nos. 4,452,775; 5,239,660; and 3,854,480).

A composition provided herein (e.g., a pharmaceutical composition) can include an acyl amino acid in association with one or more non-toxic, pharmaceutically acceptable carriers and/or diluents and/or adjuvants and/or excipients, collectively referred to herein as “carrier” materials, and if desired other active ingredients. Compositions may contain common carriers and excipients. For example, in some embodiments, a composition includes one or more of corn starch, gelatin, lactose, sucrose, microcrystalline cellulose, kaolin, mannitol, dicalcium phosphate, sodium chloride, alginic acid, or sodium starch glycolate. In some embodiments, a composition includes one or more binders, e.g., acacia, methylcellulose, sodium carboxymethylcellulose, polyvinylpyrrolidone (Povidone), hydroxypropyl methylcellulose, sucrose, starch or ethylcellulose. In some embodiments, a composition includes one or more lubricants. Lubricants include, for example, metallic stearates such as magnesium stearate, stearic acid, silicone fluid, talc, waxes, oils, colloidal silica, etc.

In some embodiments, a composition for oral use is a solid formulation such as a tablet or capsule. Sustained release or enterically coated preparations may also be devised. In some embodiments, a composition for oral use is in the form of, for example, a suspension or liquid. A pharmaceutical composition can be made in the form of a dosage unit containing a therapeutically effective amount of an active ingredient (i.e., acyl amino acid). Examples of such dosage units are tablets and capsules. A tablet or capsule can include, in addition to an active ingredient, carriers such as binding agents, for example, acacia gum, gelatin, polyvinylpyrrolidone, sorbitol, or tragacanth; fillers, for example, calcium phosphate, glycine, lactose, maize-starch, sorbitol, or sucrose; lubricants, for example, magnesium stearate, polyethylene glycol, silica, or talc; disintegrants, for example, potato starch, flavoring or coloring agents, or acceptable wetting agents. Oral liquid preparations can be aqueous or oily solutions, suspensions, emulsions, syrups or elixirs, and may contain conventional additives such as suspending agents, emulsifying agents, non-aqueous agents, preservatives, coloring agents and flavoring agents. Examples of additives for liquid preparations include acacia, almond oil, ethyl alcohol, fractionated coconut oil, gelatin, glucose syrup, glycerin, hydrogenated edible fats, lecithin, methyl cellulose, methyl or propyl parahydroxybenzoate, propylene glycol, sorbitol, or sorbic acid.

In some embodiments, an acyl amino acid composition is formulated for intravenous (IV) use. Such a composition can be dissolved or suspended in an intravenous fluid and administered by infusion. Intravenous fluids include, for example, physiological saline and Ringer's solution. Any suitable device can be used for intravenous administration. For example, intravenous administration may be with a syringe, minipump or intravenous line.

In some embodiments, an acyl amino acid composition is formulated for parenteral administration. In some embodiments, a parenteral formulation is in the form of an aqueous or non-aqueous isotonic sterile injection solution or suspension. Solutions or suspensions can be prepared from sterile powders or granules having one or more of the carriers mentioned for use in formulations for oral administration. Acyl amino acids and other components can be dissolved in substances such as polyethylene glycol, propylene glycol, ethanol, corn oil, benzyl alcohol, sodium chloride, and/or various buffers.

In some embodiments, an acyl amino acid composition is formulated for intramuscular administration. In some such embodiments, a sterile formulation of an acyl amino acid composition is dissolved and administered in a pharmaceutical diluent such as Water-for-Injection (WFI), physiological saline or 5% glucose. A suitable insoluble form of a composition may be prepared and administered as a suspension in an aqueous base or a pharmaceutically acceptable oil base.

A dose of an intravenous, intramuscular or parental formulation of an acyl amino acid composition may be administered as a bolus (e.g., a dose that is administered in less than 30 minutes) or by slow infusion. In some embodiments, a bolus is administered in less than 15 or less than 10 minutes. In some embodiments, a bolus is administered in less than 5 minutes. In some embodiments, a bolus is administered in one minute or less. In some embodiments, an infusion is a dose that is administered at a rate of 30 minutes or greater. In some embodiments, an infusion is administered for one hour or longer. In another embodiment, the infusion is substantially constant.

In some embodiments, an acyl amino acid composition is formulated for topical use. In some such embodiments, an acyl amino acid composition is prepared in a form suitable for application to skin, or mucus membranes of the nose and throat, and can take the form of creams, ointments, liquid sprays or inhalants, lozenges, and/or throat paints. Such topical formulations further can include chemical compounds such as dimethylsulfoxide (DMSO) to facilitate surface penetration of the active ingredient.

In some embodiments, an acyl amino acid composition is formulated for administration to eyes or ears. For example, an acyl amino acid composition can be presented in liquid or semi-liquid form formulated in hydrophobic or hydrophilic bases as ointments, creams, lotions, paints or powders. For rectal administration, an acyl amino acid composition can be administered in the form of suppositories admixed with conventional carriers such as cocoa butter, wax or other glyceride. Alternatively, an acyl amino acid composition can be in powder form for reconstitution in the appropriate pharmaceutically acceptable carrier at the time of delivery. In some embodiments, a unit dosage form of the composition can be a solution of the composition in a suitable diluent in sterile, hermetically sealed ampoules or sterile syringes. The concentration of an acyl amino acid in the unit dosage may vary, e.g. from about 1 percent to about 50 percent, depending on the particular acyl amino acid used and its solubility and the dose desired by the physician. If compositions contain dosage units, each dosage unit can contain from 0.1-500 mg of the active material (i.e., acyl amino acid). In some embodiments for adult human treatment, a dosage employed ranges from 0.5 mg to 10 g, per day, depending on the route and frequency of administration.

Also provided herein are methods for inhibiting the growth of microbes, e.g., bacteria. The methods include contacting the microbes with an acyl amino acid (e.g., beta hydroxyl myristoyl glutamate). The present disclosure further includes compositions including acyl amino acids in an amount sufficient to reduce the presence of microbes (e.g., bacteria, e.g., S. aureus) on an object. Methods of using acyl amino acids to enhance the antimicrobial effectiveness of a composition are also provided. The methods include adding to the composition an amount of an acyl amino acid (e.g., beta hydroxyl myristoyl glutamate) effective to kill bacteria.

### Examples

### Example 1

### Purification of Fatty Acyl Glutamate (FA-Glu)

In some embodiments, an engineered microbial strain is used to provide an acyl amino acid. For example, any strain that produces FA-Glu, e.g., by strain engineering, can be employed for production of this acyl amino acid. In some embodiments, the strain OKB105 Δ(upp)Spect^{R} FA-GLU-TE, which is described in PCT/US08/60474, published as WO2008/131002, is employed for production of FA-Glu. FA-Glu can be recovered from any aqueous media, such as M9YE supplemented with 0.5% glucose and 1% casamino acids.

For the initial step of producing FA-Glu, media (cells included) of FA-Glu expressing cells was adjusted to pH to 8 with sodium hydroxide to ensure that all FA-Glu produced is in soluble form. The media was filtered through an ultrafiltration apparatus (GE Healthcare) with a membrane cutoff of 500 k Da. The filtered material was then adjusted to pH 7 with hydrochloric acid and passed through a Discovery Bio Wide pore C18 HPLC column (5 cm×21.2 mm, 10 μm) using a gradient of increasing acetonitrile concentration from 0% to 100%. Buffer A is water. Fractions were collected and those that contain FA-Glu, regardless of the length of the fatty acid chain, were pooled and dried. FA-Glu was resuspended in water at pH 9.5 and extracted with butanol. The addition of 1.5M NaCl to the aqueous solution facilitated extraction of FA-Glu into the butanol phase. The butanol phase was washed twice with water at pH 2 to remove salt and proteins present in the aqueous phase near the aqueous-butanol interface. Then, the butanol, containing FA-Glu, was dried, resuspended in water and the pH adjusted to 7. This mixture was passed through a Discovery Bio Wide pore C18 HPLC column (5 cm×21.2 mm, 10 μm) using a gradient of increasing acetonitrile concentration from 0% to 100%. Buffer A is water. Fractions were collected and those that contain FA-Glu, regardless of the length of the fatty acid chain, were pooled and dried.

### Example 2

### Analysis of Antimicrobial Activity of FA-Glu

The ability of FA-Glu to inhibit the growth or kill S. aureus (ATCC 6538) was examined. The data herein show by disk assay that FA-Glu prevents the growth of this organism.

S. aureus was grown in liquid culture in either Tryptic Soy Broth (TSB; Sigma), which contains casein peptone 17 g/l, soya peptone 3 g/l, sodium chloride 5 g/l, dipotassium hydrogen phosphate 2.5 g/l, and glucose 2.5 g/l or M9YE (disodium hydrogen phosphate 6 g/l, monopotassium phosphate 3 g/l, sodium chloride 0.5 g/l, ammonium chloride 1 g/l, yeast extract 3 g/l, glucose 5 g/l, and casamino acids 10 g/l) at 30° C. Disk assays were carried out on M9YE plates (M9YE liquid media plus 15 g/l agar) and YPD plates (peptone 20 g/l, yeast extract 10 g/l, glucose 20 g/l, agar 15 g/l). S. aureus was plated out evenly using a sterile cotton swab. Blank Paper disks 6 mm were placed into a sterile container and then different dilutions of FA-Glu (25 ug, 100 ug, 300 ug) that had been re-suspended in a diluted aqueous solution containing ammonium hydroxide (pH 10.2) were added to the disks. The disks were allowed to absorb and dry. Using sterile tweezers, the disks were placed onto the plates that had been swabbed with Staphylococcus aureus. They were then incubated at 28° C. over approximately 65 hours. A clear and defined halo with no cell growth was formed around the 300 ug disk and not around disks having any other concentration. No halo was visible on the disk containing the solution used to resuspend FA-Glu. The halo produced by the 300 ug of FA-Glu was present for 12 days with no sign of deterioration. This experiment was repeated at 37° C. with similar results, namely, no cell growth around the 300 ug disk, after 9 days. The halo effect produced by FA-Glu was more pronounced on YPD plates than on M9YE plates. A halo effect was also observed using FA-Glu on S. aureus plated on TSB. This effect was less pronounced than seen on M9YE plates.

In a control experiment, 300 ug of surfactin absorbed onto a disk produced a halo, but there was cell growth within the halo.

Disk assays were repeated as above using a commercially available acyl amino acid, cocoyl glutamate (Ajinomoto, Japan). Cocoyl glutamate also produced a halo. Halos were larger than halos observed using FA-Glu at the same concentration and under the same conditions. However, regrowth occurred in halos produced by cocoyl glutamate. Regrowth was not observed with FA-Glu-treated plates.

What is claimed is:

1. A method of treating a bacterial infection in a subject, the method comprising administering to the subject a therapeutically effective amount of a pharmaceutical composition comprising an antibacterial acyl amino acid, wherein the acyl amino acid comprises a fatty acid.

2. The method of claim 1, wherein the bacterial infection comprises a Staphylococcus infection.

3. The method of claim 2, wherein the Staphylococcus infection comprises an infection by Staphylococcus aureus.

4. The method of claim 3, wherein the Staphylococcus aureus comprises an antibiotic resistant strain.

5. The method of claim 4, wherein the antibiotic strain is resistant to one or more of methicillin, amoxicillin, penicillin, and amoxicillin.

6. The method of claim 1, wherein the subject is receiving, or has received antibiotic therapy for the infection.

7. The method of claim 1, wherein the subject is a human.

8. The method of claim 1, wherein the subject is a non-human animal.

9. The method of claim 1, wherein the fatty acid comprises a beta-hydroxy fatty acid.

10. The method of claim 9, wherein the beta-hydroxy fatty acid comprises a fatty acid chain which comprises 3-20 carbon atoms.

11. The method of claim 10, wherein the fatty acid chain of the beta-hydroxy fatty acid comprises 13-15 carbon atoms.

12. The method of claim 1, wherein the fatty acid comprises a saturated fatty acid selected from butryic acid, caproic acid, caprylic acid, capric acid, lauric acid, myristic acid, palmitic acid, stearic arachidic acid, behenic acid, and lignoceric acid.

13. The method of claim 1, wherein the fatty acid comprises an unsaturated fatty acid selected from myristoleic acid, palmitoleic acid, oliec acid, linoleic acid, alpha-linolenic acid, arachidonic acid, eicosapentaenoic acid, erucic acid, and docosahexaenoic acid.

14. The method of claim 1, wherein the acyl amino acid comprises glutamate.

15. The method of claim 1, wherein the acyl amino acid comprises beta hydroxyl myristoyl glutamate.

16. The method of claim 1, wherein the composition is administered topically.

17. The method of claim 1, wherein the composition is administered to a mucosal surface.

18. The method of claim 17, wherein the composition is administered orally.

19. The method of claim 17, wherein the composition is administered intranasally.

20. The method of claim 1, wherein the composition is administered parenterally.

21. The method of claim 20, wherein the composition is administered intravenously.

22. The method of claim 20, wherein the composition is administered subcutaneously or intramuscularly.

23. The method of claim 1, wherein the composition comprises the acyl amino acid and a pharmaceutically acceptable carrier.

24. The method of claim 1, wherein the composition consists essentially of the acyl amino acid and a pharmaceutically acceptable carrier.

25. The method of claim 1, wherein the composition is administered to the subject over 7 or more days.

26. A method of reducing risk for bacterial infection in a subject, the method comprising administering to the subject a pharmaceutical composition comprising an antibacterial acyl amino acid, wherein the acyl amino acid comprises a fatty acid, and wherein the acyl amino acid is present in an amount sufficient to kill bacteria in the subject.

27. The method of claim 26, wherein the subject is a subject at risk for exposure to Staphylococcus aureus.

28. The method of claim 27, wherein the subject is at risk for nosocomial infection by Staphylococcus aureus.

29. The method of claim 26, wherein the subject is a human.

30. The method of claim 26, wherein the subject is a non-human animal.

31. The method of claim 26, wherein the fatty acid comprises a beta-hydroxy fatty acid.

32. The method of claim 31, wherein the beta-hydroxy fatty acid comprises a fatty acid chain which comprises 3-20 carbon atoms.

33. The method of claim 32, wherein the fatty acid chain of the beta-hydroxy fatty acid comprises 13-15 carbon atoms.

34. The method of claim 26, wherein the fatty acid comprises a saturated fatty acid selected from butryic acid, caproic acid, caprylic acid, capric acid, lauric acid, myristic acid, palmitic acid, stearic arachidic acid, behenic acid, and lignoceric acid.

35. The method of claim 26, wherein the fatty acid comprises an unsaturated fatty acid selected from myristoleic acid, palmitoleic acid, oliec acid, linoleic acid, alpha-linolenic acid, arachidonic acid, eicosapentaenoic acid, erucic acid, and docosahexaenoic acid.

36. The method of claim 26, wherein the acyl amino acid comprises glutamate.

37. The method of claim 26, wherein the acyl amino acid comprises beta hydroxyl myristoyl glutamate.

38. The method of claim 26, wherein the composition is administered topically.

39. The method of claim 26, wherein the composition is administered to a mucosal surface.

40. The method of claim 39, wherein the composition is administered orally.

41. The method of claim 39, wherein the composition is administered intranasally.

42. The method of claim 26, wherein the composition is administered parenterally.

43. The method of claim 42, wherein the composition is administered intravenously.

44. The method of claim 42, wherein the composition is administered subcutaneously or intramuscularly.

45. The method of claim 26, wherein the composition comprises the acyl amino acid and a pharmaceutically acceptable carrier.

46. The method of claim 26, wherein the composition consists essentially of the acyl amino acid and a pharmaceutically acceptable carrier

47. A pharmaceutical composition comprising an antibacterial acyl glutamate and a pharmaceutically acceptable carrier, wherein the acyl glutamate comprises a beta-hydroxy fatty acid, and wherein the acyl glutamate is present in an amount sufficient to kill Staphylococcus aureus.

48. The composition of claim 47, which is formulated for administration to a mucosal surface.

49. The pharmaceutical composition of claim 48, which is formulated for oral administration.

50. The pharmaceutical composition of claim 48, which is formulated for intranasal administration.

51. The pharmaceutical composition of claim 47, which is formulated for parenteral administration.

52. The pharmaceutical composition of claim 47, which is formulated for topical administration.

53. A disinfectant composition comprising an antibacterial acyl glutamate and a carrier, wherein the acyl glutamate comprises a beta-hydroxy fatty acid, and wherein the acyl glutamate is present in an amount sufficient to kill Staphylococcus aureus.


SEQUENCE LISTING (2 sequences)

SEQ ID NO: 1 - protein, 97 aa, Artificial Sequence
  Synthetic peptide
LIVSPADNKXXXXXXXXXPXXYPSTAGNCVSTAGNQCIVMSTAGCKPCSAGFYRLIVMSTAGDXKLIVMFYWDXYRLIVMFYWGAPTHQGSACQRHM

SEQ ID NO: 2 - protein, 31 aa, Artificial Sequence
  Synthetic peptide
LIVKGLIVFYLIVMSTGHYWVSYAGGGSTAC